(19) United States
(12) Patent Application Publication
(10) Pub. No.: US 2005/0177129 A1
(43) Pub. Date: Aug. 11, 2005

(54) Immunization system

(76) Inventors: Ted H. Pacha, Iowa City, IA (US); Della Jean Schroeder, Maquoketa, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 10/773,926

(22) Filed: Feb. 6, 2004

(51) Int. Cl. A61M 31/00

(52) U.S. Cl. 604/500; 604/189; 602/42

(57) ABSTRACT

A method of immunization identification comprising filling a first syringe with a first immunization dosage, applying a first adhesive-backed strip having a first color to the first syringe, injecting the first dosage into a patient at a first injection site, applying a second adhesive-backed strip having a first color to the first injection site after the injection and applying a third adhesive-backed strip having the first color to a medical office chart of the patient. An immunization identification system, comprising a first series of stickers all having a first color corresponding to a first immunization, and a second series of stickers all having a second color corresponding to a second immunization. The first and second series of stickers being adapted to be applied to first and second syringes, to first and second patient injection sites, and to a medical chart of the patient.

### BACKGROUND OF THE INVENTION

People are interested in having active knowledge of what medications/vaccines are being administered to them or their family members. They should be able to accurately watch for and report any negative reactions to the medication/immunization given to them. Currently there is a need for an organized universal protocol to make the delivery, documentation and follow-up of immunization systems simple, accurate and reliable processes.

Currently every child in the United States is required by law to have a total of 19 immunizations from birth to kindergarten. There is a recommended sequence with different immunizations being given at ages 2 months, 4 months, 6 months, 12 months, 15 months, and 18 months, and booster shots given between 4 and 6 years of age. This becomes complicated when children do not get their immunizations on schedule (at the recommended ages) due to illness or lack of compliance on the part of the parent or care givers. It also becomes more confusing when two or more children of different ages are brought to the clinic to receive immunizations at the same time. There is further confusion when a caregiver brings in two children who have different insurance situations due to paternity. The process may also become more complex when people move and change doctors.

Oftentimes it is necessary for the nurse to prepare six to eight immunizations (syringes) for injection in the same exam room. Currently all of the childhood immunizations are given either intra-muscularly (IM) or subcutaneously (SQ) depending upon which method facilitates the best absorption into the body. The immunizations are produced by many different pharmaceutical companies with each manufacturer having a different label and different colored vial cap and a different brand name. To complicate the process even more, there are two different sets of vaccines to be used, which are kept on a refrigerator shelf often side-by-side. One supply is for patients who have medical insurance coverage of their own and the other supply is used for patients who have no insurance coverage or are on a government sponsored insurance program such as Medicaid. This supply, provided by the government, may come from a different manufacturer each time. The color of the vial cap and the brand name of the immunization may be different with each new supply. This makes identification of contents in the vial difficult at a quick glance.

Once the immunizations are drawn from the vial into the syringe, there is nothing to distinguish one immunization from the other unless the nurse labels the syringes with a marker of some type. The next step of the process is to carry the syringes to the exam room and prepare to inject them into various sites on the child's thighs. Currently, after each injection is completed, the site is covered with a nondescript bandage. It is highly recommended that the specific injection site of each immunization be documented in the patient's permanent record or chart. With multiple injections, crying children and upset parents, it can be very difficult for the nurse to remember which immunization was given at which site. It is important to know the location of each specific immunization injection site, in case the recipient should develop a reaction to the ingredients in one of the immunizations administered. The next step is to fill out the patient's immunization record in the permanent file chart with the proper information including the manufacturer's lot number, the expiration date of the medication and the date the immunization was administered. Most parents also will want to keep a home record of the child's immunizations, thus making another area necessary for the nurse to chart the type and date the immunizations were given.

If a different child is immunized every fifteen minutes, receiving four injections each, that totals 16 injections per hour, with 4 hours per am or pm shift. Thus, potentially 64 immunizations could be required to be administered and charted per shift by one nurse. This is an enormous amount of charting injection sites, lot numbers and expiration dates, while calming fears, wiping tears and instructing parents on side effects that are normal and abnormal. In view of these numerous factors, some innocent errors are made in the injection and documentation process in any given morning or afternoon.

### SUMMARY OF THE INVENTION

A method of immunization identification and a coded immunization system is provided for simple, accurate and reliable processing. The code may be a color, a shape, a number, a letter, a picture or any combination thereof so as to be universally recognized as specific to that particular immunization.

This immunization identification system consists of a universally coded strip or bandage and identification stickers to be used at the time the immunizations are administered and documented. Each dose, whether a prefilled syringe, a single unit dose, or a multi-dose vial is accompanied by the corresponding number of identification strips per package (i.e., five doses per package equals five identification strips per package). Ideally, the vial caps, regardless of manufacturer, will all be coded to match the identification strip (i.e., DtaP (diphtheria, tetanus and pertussis vaccine) equals blue, MMR (measles, mumps and rubella vaccine) equals red, etc.). This coded concept will be universally applied and recognized as each specific vaccine's identifiable code. The immunization strip contains:

(a) a small coded adhesive tab to be applied to the individual syringe at the time the nurse draws up the vaccine for a specific patient and readily identifiable by code when she is ready to inject each immunization. A syringe adhesive tab is preferably preprinted with an accepted vaccine abbreviation (DtaP, IPV (inactivated poliovirus vaccine), MMR) and the preferred method of injection (IM or SQ).

(b) two or more coded adhesive tabs to be applied to the patient's chart and to the patient's home record. The tab that is applied to the chart is preferably preprinted with the manufacturer's lot number and expiration date. If not preprinted, the nurse writes this information onto the colored tab or label. There is also space for a nurse to record the injection site (i.e., left lateral thigh, upper or right lateral thigh, lower, etc.). This adhesive tab (label) can be readily spotted in the chart, due to its identifiable code, at the time of administering vaccine and in the future when filling out immunization records for school, travel, or when auditing records, etc.

(c) a coded bandage strip to be applied to the patent's immunization site. This allows the nurse to glance back at the injection sites upon charting, insuring that the accurate injection site is recorded. Preferably, the abbreviation of the name of the immunization is also on the bandage strip. In the event of a unexpected adverse reaction appearing at the site of the injection, caretakers can easily recognize which immunization was responsible for the reaction and thus notify the healthcare facility at which the vaccination was performed.

(d) a bar code to be used for patient charges and/or computer charting. Bar codes could be used by more technologically advanced facilities and additional facilities in the future when those charting a patient file could be done on the computer.

### BRIEF DESCRIPTION OF THE DRAWINGS

FIG. 1 is a top view of an immunization identification strip of the present invention.

FIG. 2 is a diagram that shows the application of all the component parts of the immunization identification strip of the present invention.

FIG. 3 is a flow chart that shows the steps involved in the immunization process according to the present invention.

### DETAILED DESCRIPTION OF THE PREFERRED EMBODIMENT

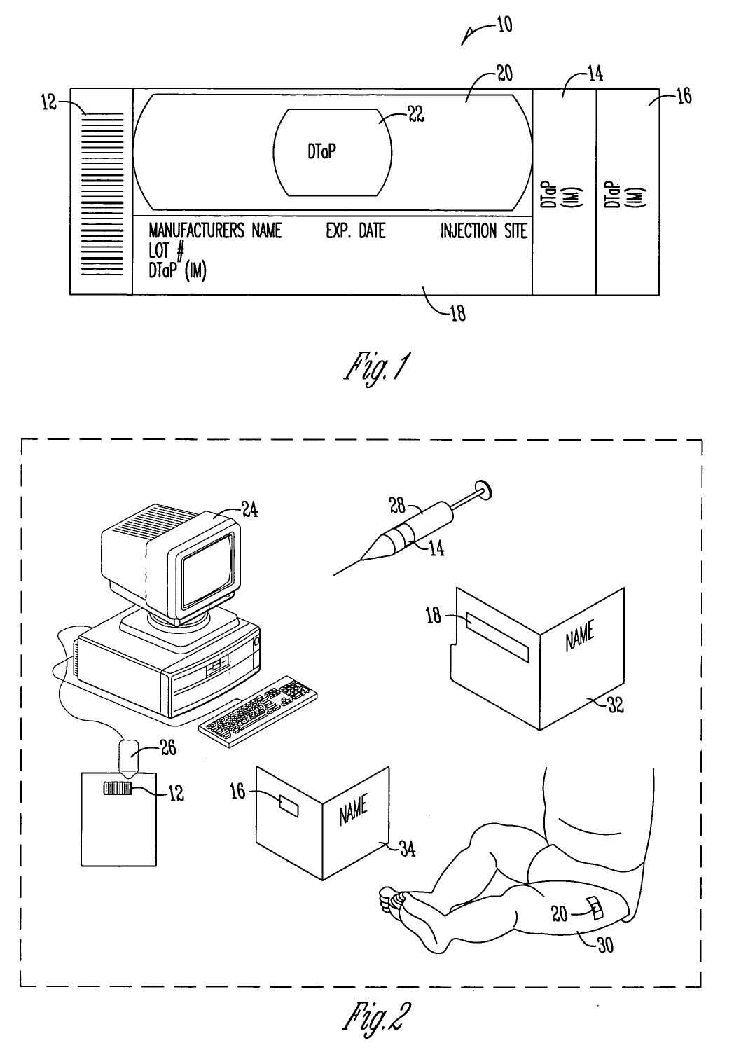

Referring to FIG. 1, reference numeral 10 generally refers to the immunization identification strip of the present invention. The adhesive-backed strip 10 is made up of component stickers: a bar code sticker 12, a syringe sticker 14, a home medical record sticker 16, a medical chart sticker 18, and a bandage sticker 20. The bar code sticker 12 is optional, and contains information that may be uploaded into a computer after scanning with a bar code reader 26. At a minimum, the name of the vaccine and the method of injection are printed on the syringe sticker 14 and home medical record 16. The medical chart sticker 18 is for the doctor's records and contains additional information including the vaccine manufacturer's name, the vaccine lot number, the expiration date of the vaccine and the injection site in addition to the name of the vaccine and the method of injection. Gauze 22 is attached to the middle underside of the bandage sticker 20. The gauze 22 is to be applied directly over the injection wound. The name of the vaccine is printed on the front of the bandage sticker 20.

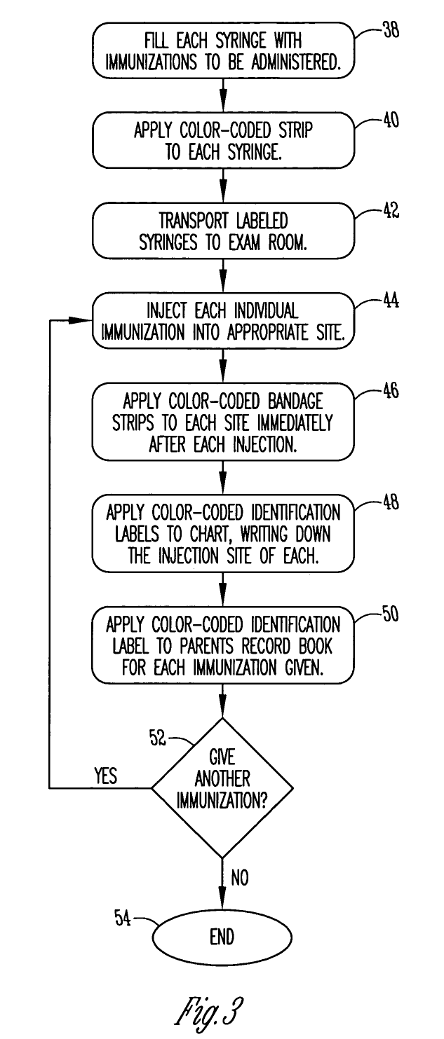

FIG. 2 schematically shows the use of the stickers 12-20 of the immunization identification strip 10. The bar code sticker 12 is read by a bar code reader 26, which is in communication with a computer 24. The syringe sticker 14 is placed on the syringe 28. The home medical record sticker 16 is attached to the patient's home record book 34. The medical chart sticker 18 is attached to the patient's chart 32. The bandage sticker 20 is applied to the patient 30.

FIG. 3 shows a flowchart for the immunization identification method according to the present system. Following the flow chart, the first step 38 is filling the syringes 28 with the immunization dosages. This is followed by the step 40 of applying a colored strip to the syringes 28 and then the step 42 of transporting the labeled syringes 28 to the room where the patient is waiting. The next step 44 is injecting the immunizations into a patient. Then colored strips are applied to the injection sites, as indicated by step 46, followed by the steps 48 and 50 of applying colored strips to the patient's charts 32 and 34. Steps 44 through 50 are repeated until all the immunizations required at that visit are completed for that particular patient, as indicated by decision steps 52, 54. It is noted that the order of the steps 38-50 may be modified without departing from the scope of the invention.

This methodology and identification system facilitates the tracking of immunizations, patterns of adverse reactions and gives the parent peace of mind, knowing exactly what kind of immunization was delivered and at what site. With the development of a coded instruction and record book, showing an example of a properly completed immunization record, the medical professionals and parent knows how many immunizations a child has received to date and how many are still necessary to complete the series of immunizations required by state law.

As new immunizations are being developed and new combinations are being administered simultaneously, it is extremely critical that an accurate procedure is followed, accurate records are kept, and adverse reactions are reported to healthcare providers and immunization manufacturers. There are great benefits to this organizational universal system with the coded strip 10 to alleviate the chance of error or omission especially when more than one medication/immunization is administered at the same time. If vaccine manufacturers find this coded strip 10 to be useful, it is expected that color coding the vial caps to match the strips 12-20 would be a benefit, thus making this a complete universally coded product and system.

There are several possibilities of how these immunization identification systems could be distributed. These possibilities include, but are not limited to, the vaccine (drug) manufacturing companies, syringe manufacturers and bandage strip manufacturers.

A coded system has strong positive implications for the accuracy of the U.S. Immunization System. A similar coded system may also be applied to other medical situations where a series of drugs or treatments need to be administered, documented and followed-up (tracked) in a systematic manner. However, once a certain code is assigned to a particular immunization medication or treatment, it should stay exclusive to that medication or treatment and another type of code would be exclusive to each particular unique medical procedure being coded and documented.

In the drawings and specification, there has been set forth a preferred embodiment of the invention, and although specific terms are employed, their use is in a generic descriptive sense only and not for purposes of limitation. Changes in the form and their proportion or parts, as well as in the substitution of equivalents are contemplated as circumstance may suggest or render expedient without departing from the spirit or scope of the invention in the following claims.

1. A method of immunization identification, comprising:
filling a first syringe with a first immunization dosage;
applying a first adhesive-backed strip having a first color to the first syringe;
injecting the first dosage into a patient at a first injection site;
applying a second adhesive-backed strip having a first color to the first injection site after the injection; and
applying a third adhesive-backed strip having the first color to a medical office chart of the patient.

2. The method of claim 1 further comprising:
filling a second syringe with a second immunization dosage;
applying a fourth adhesive-backed strip having a second color to the second syringe;
injecting the second dosage into the patient at a second injection site;
applying a fifth adhesive-backed strip having the second color to the second injection site after the injection; and
applying a sixth adhesive-backed strip having the second color to the medical office chart of the patient.

3. The method of claim 1 further comprising applying a fourth adhesive-backed strip having the first color to a home chart of the patient.

4. The method of claim 1 further comprising providing the first color on a first vial containing the first immunization dosage.

5. The method of claim 4 further comprising providing the second color on a second vial containing the second immunization dosage.

6. A method of identifying immunizations given to a patient, comprising: applying a first series of stickers corresponding to a first immunization to a first syringe, a first injection site, and a medical record chart of a patient, the first series of stickers all having a common first indicia.

7. The method of claim 6 further comprising applying a second series of stickers corresponding to a second immunization to a second syringe, a second injection site, and the medical record of the patient, the second series of stickers having a common second indicia.

8. The method of claim 7 wherein the first and second indicia are first and second colors.

9. The method of claim 7 wherein the first and second series of stickers are further applied to a home chart of the patient.

10. The method of claim 7 further comprising providing the first and second indicia on first and second vials containing the first and second immunizations, respectively.

11. An immunization identification system, comprising:
a first series of stickers all having a first color corresponding to a first immunization; and
a second series of stickers all having a second color corresponding to a second immunization;
the first and second series of stickers being adapted to be applied to first and second syringes, to first and second patient injection sites, and to a medical chart of the patient.

12. The immunization identification system of claim 11 wherein each of the first and second series of stickers includes at least three stickers for the respective syringe, injection site and medical chart.

13. The immunization identification system of claim 12 wherein each of the first and second series of stickers includes a fourth sticker for a home chart of the patient.

14. The immunization identification system of claim 11 wherein each of the first and second series of stickers includes a bar code for billing a payor.

15. The immunization identification system of claim 11 wherein each sticker includes a written identification of the immunization type.
